(19) United States
(12) Patent Application Publication
(10) Pub. No.: US 2012/0253233 A1
(43) Pub. Date: Oct. 4, 2012

(54) ALGORITHM FOR QUANTITATIVE STANDING BALANCE ASSESSMENT

(76) Inventors: Barry GREENE, Dublin (IE); Lorcan WALSH, Staplestown (IE); Cliodhna NISCANAILL, Chorcai (IE)

(21) Appl. No.: 13/198,343

(22) Filed: Aug. 4, 2011

Related U.S. Application Data
(60) Provisional application No. 61/470,453, filed on Mar. 31, 2011.

(51) Int. Cl. A61B 5/103; A61B 5/11

(52) U.S. Cl. 600/592

(57) ABSTRACT

A system, method, and apparatus is provided for calculating a risk of falls from pressure measurements. Pressure data may be measured by a pressure sensor matrix, such as a pressure mat. Multiple snapshots of pressure data may be generated, each with a plurality of pressure points corresponding to coordinates in the pressure sensor matrix. Pressure-based metrics, including center of mass and center of pressure, may be derived from the pressure measurements. The plurality of centers of pressure and other pressure-related parameters may be used to generate a statistical model that predicts the risk of falls.

This application claims priority to U.S. Provisional Patent Application Ser. No. 61/470,453, filed Mar. 31, 2011, the entire content of which is incorporated herein by reference.

### FIELD OF THE INVENTION

The invention relates to sensing devices and methods that may be used to quantitatively measure balance and postural stability based on pressure sensor data.

### BACKGROUND OF THE INVENTION

Falls have been considered a “geriatric giant” and are associated with negative health outcomes such as serious injury, hospitalization, restricted mobility, and institutionalization. Falls have a negative effect on quality of life, lead to increased hospitalization, and are costly. The cost of falls each year among the elderly in the U.S. alone has been estimated to be about $20 billion. Falls in older adults are common and their incidence increases with age.

Postural stability and balance has been associated with falls amongst older adults. As people age, changes in gait, strength, and sensory abilities may lead to a decline in the person's posture and balance. Methods to measure balance and postural stability have been performed in clinical settings involving force plates or optical motion capture systems that measure a patient's center of pressure (COP) or postural sway during a standing exercise. Such techniques have been expensive because of the need for clinical visits and specialized equipment and trained personnel.

### SUMMARY OF THE INVENTION

One aspect of the invention relates to a system and method for measuring balance and postural stability based on pressure data. A portable pressure sensor matrix comprising pressure sensors may be used to measure a person's pressure distribution as the person stands on the mat. Changes in the pressure distribution, such as from a person's shifting his or her weight, may also be recorded. The measurement system may be portable, and pressure measurement may be done in a clinical setting or in the home. For example, a measurement of postural stability may be done in a home environment with a pressure sensor matrix, without requiring supervision from specially trained personnel. This unsupervised assessment may reduce the cost of falls assessment and facilitate the gathering of data in a longitudinal (e.g., daily) monitoring of falls risk.

The measurement may be done in combination with standard tests such as the “timed up and go” (TUG) test or the Berg balance scale (BBS), allowing the data to be integrated to a standard clinical assessment of a person's postural stability and/or risk of falling. The measurements may include any other test that measures pressure using the pressure sensor matrix. The measurements may be processed locally, by components within the pressure sensor matrix, or may be processed by a remote processor or server that is configured to communicate with the pressure sensor matrix via a wired or wireless interface.

The pressure data may be used to assess balance and postural stability based on statistical models relating pressure to stability. The pressure data may be used to specifically derive measures of plantar pressure, heel/toe and mid-foot pressure variation, center of pressure, center of mass metrics, or any other metrics related to balance. The pressure data may be used to extract a planar fit of the pressure values and locations on the sensor matrix associated with pressure exerted by a person's heels and toes. The data derived using the system and method described herein may be used to classify falls risk based on features derived from the balance test. For example, supervised or unsupervised pattern recognition may be used to determine a risk of future falls from the measured metrics that relate to balance. The timely determination of falls risk would facilitate appropriate intervention, such as a tailored balance and strengthening program, that would reduce the risk of future falls.

In one embodiment, the pressure sensor matrix may include a high density pressure sensitive floor mat having a plurality of sensors that collect pressure data generated from the presence of a person on the mat. Pressure data may be binary, such as the presence or absence of a threshold pressure, or may have more granular values corresponding to the amount of pressure. The pressure data may be collected at a plurality of times. The data corresponding to one of the plurality of times may make up a series of time samples (or snapshots) of pressure data.

One or more processors located on the pressure mat or at a remote location may implement program modules to process the pressure data. The modules may decide to process the data only if a sufficient number of data points was collected from the floor mat or from some other pressure sensor matrix.

The modules may calculate a center of pressure (COP) from the pressure data. In one embodiment, the COP may be an average position of the points of pressure detected by the pressure sensor matrix. The average position may be weighted based on pressure values at each point of pressure. For example, the COP may be shifted to the left based on higher pressure values on the left side of the pressure sensor matrix. In one embodiment, pressure data points from the pressure data may be divided into regions corresponding to the heel, toe, and/or mid-foot locations of the user who exerted the pressure on the matrix. The regions may be identified for both a left foot and right foot. The centroid of the pressure points of each or some of the (e.g., of the toe and heel) regions may be identified based on an average of the pressure points in the corresponding region. Each pressure point may be weighted based on its pressure value. The COP may then be calculated as the average position of each of the regional pressure centroids. For example, the positions of pressure centroids of the left heel region, left toe region, right heel region, and right toe region may be averaged to yield the COP. The centroid of the pressure points of a region may be weighted based on a pressure value of the region, such as a mean pressure, maximum pressure, minimum pressure, or any other pressure-related value.

The COP may be calculated for each snapshot to produce a COP time series that corresponds to measurements taken over the duration of a balance assessment test. Standard time and frequency domain measures for quantifying the center of pressure may be used to quantify the data obtained during the assessment.

In one embodiment, kinematic (inertial) sensors may also be used with the pressure sensor matrix. For example, an accelerometer, gyroscope, or magnetometer may be used to collect data on the movement of a user. The collected pressure and kinematic data may be combined and used to predict falls risk in a user.

### BRIEF DESCRIPTION OF THE DRAWINGS

FIG. 1A illustrates an example setup in which pressure data for assessing postural stability is acquired.

FIG. 1B illustrates an example graphical view of data collected by a pressure sensor matrix.

FIG. 2 illustrates example operations that may be performed to generate pressure-based standard balance metrics and to generate a classification of falls risk.

FIG. 3A illustrates an example graphical view of data collected by a pressure sensor matrix.

FIG. 3B illustrates an example planar fit of four pressure values and locations corresponding to toe and heel pressure generated by a balance assessment test participant.

FIG. 4 illustrates example operations that may collect center of pressure data for assessing postural stability.

FIG. 5 illustrates a graphical view of example pressure-related data collected by a pressure sensor matrix and their relation to a calculated center of pressure.

FIG. 6 illustrates a graphical view of example measurements of centers of pressure from a plurality of pressure snapshots.

FIG. 7 illustrates a graphical view of example measurements of centers of pressure, presented relative to an anteroposterior axis and a mediolateral axis, from a plurality of pressure snapshots.

FIG. 8 illustrates a graphical view of example measurements of centers of pressure, presented as a function of time, from a plurality of pressure snapshots.

FIG. 9 illustrates a graphical view of metrics related to postural stability.

FIG. 10A illustrates an example relationship of center of pressure metrics derived from a force plate setup and from a pressure mat setup.

FIG. 10B illustrates an example relationship of center of pressure metrics derived from a force plate setup and from a pressure mat setup.

FIG. 11 illustrates a user interface for performing and displaying data relating to postural stability and falls risk assessment.

### DETAILED DESCRIPTION

One aspect of this invention is directed toward assessing balance and postural stability using pressure sensors. Pressure sensors may derive pressure-related data, and an algorithm may process the data to generate balance-related metrics and may generate statistical models that may predict a risk of future falls. The data gathering may be a part of a clinical balance assessment, or may be used as part of a daily or longitudinal monitoring program done in a person's home. The data gathering may be done with or without medical supervision.

The pressure sensors may be configured as a pressure sensor matrix capable of measuring pressure as a function of a plurality of coordinates that correspond to locations on the matrix. For example, the pressure sensor matrix may be a high-density pressure mat, such as the floor mat pressure sensor provided by Tactex™, which generates pressure data using KINOTEX® technology. The pressure matrix may be rigid, or may be flexible to assist in portability. The pressure sensor matrix may present an area large enough to measure how a user distributes his or her pressure over time on the matrix. For example, the matrix may be a 7′×4′ floor mat with a matrix of 3,456 sensors. The sensors may be embedded within the mat as a grid, as a staggered array, or in some other configuration. In another embodiment, the mat may be larger or smaller, and may have from a few pressure sensors to tens or hundreds of thousands of pressure sensors. The number of sensors may be adjusted based on the desired granularity of the pressure data. The number of sensors may be selected to resolve the position of an applied pressure to within a range of, for example, a few millimeters.

The pressure sensors in one embodiment may be piezoelectric pressure sensors. In one embodiment, the pressure sensors may be conductive or semiconductor material that changes resistance based on pressure or deformation. In one embodiment, the pressure sensor may be a polymer material, such as KINOTEX® polymer foam. The pressure sensors may be any material with a property that changes based on pressure or pressure-induced changes in structure. The pressure sensors may output a signal based on detecting any amount of pressure, on detecting an amount of pressure above a threshold pressure, on detecting changes in pressure in one sensor or in a threshold number of sensors, or some combination thereof. In one embodiment, the pressure sensor matrix may be placed flush on top of a force plate, which may be used as benchmark against which the accuracy or reliability of the pressure mat sensors can be compared. When comparing the data, in order to ensure that data collected is synchronized between the force plate and pressure mat (or other pressure sensor matrix), a syncing pulse may be transmitted from the force plate's computer. This signal may be captured using a dedicated sensor. The signal may be used to synchronize the data captured by the pressure mat with the data captured by the force plate.

The pressure sensors may be configured to detect only the presence of a threshold pressure, a pressure value, or a change in pressure value, or some combination thereof. For example, the pressure sensors may produce only a binary value that indicates whether the applied pressure is greater than a threshold pressure. In another example, the pressure sensor may produce a pressure value in a range from 0.1 kPa to 200 kPa, or some other range. The range of operation for the pressure sensors may be any range configured to support detecting movement, changes in posture, or changes in balance of a human or other animal. In one example, changes may be recorded when a certain number of sensors (e.g., 200) are deemed to have changed in output. In one example, changes may be recorded periodically, such as at a sampling rate of 10 Hz.

Kinematic (inertial) sensors may be incorporated into the balance assessment test to measure, for example, a test participant's gait. Kinematic sensors may include accelerometers, gyroscopes, magnetometers, global positioning system (GPS) transceivers, RFID tags, or any other sensor capable of detecting movement. For example, kinematic sensors may be sensors based on the SHIMMER™ sensor platform, which includes a 3-axis accelerometer, a battery, and electronic storage.

The pressure sensor matrix and kinematic sensors may be configured to communicate sensor data over a wired interface or over a wireless interface, such as WLAN or Bluetooth. The sensor data may be communicated to a computing platform such as a desktop, laptop, mobile phone, or other mobile device.

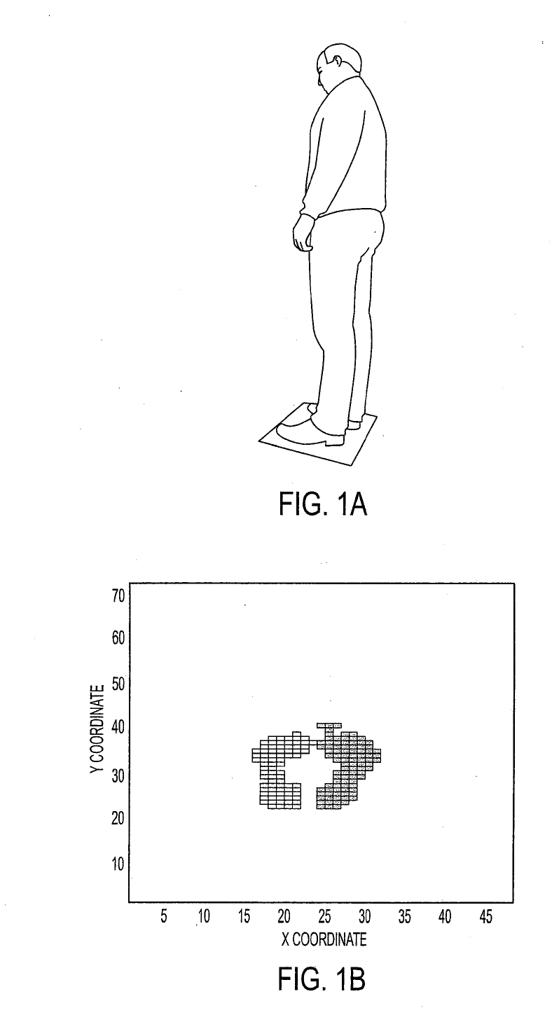

FIG. 1A illustrates pressure data being collected from a test participant. In one example, balance assessment tests may be conducted with test participants each standing still on a pressure sensing mat and facing the same direction. The participant may be instructed to remain in a comfortable stance during each balance test and may also be instructed to gaze fixed forward. The participants may hold their arms by their side, or may extend their arms outward from their bodies. The participant may stand with both eyes open or both eyes closed. Each test may last from a few seconds to a few minutes. In one example, each test lasted approximately sixty seconds, and pressure data was collected during the middle thirty seconds. Multiple tests, such as repetitions of the same balance test, may be conducted with the same test participant. In one example, there may be between one to two minutes of rest between tests. Each time a certain number (e.g., 200) of sensors are deemed to have changed, the pressure data from those or from all sensors may be captured. An example snapshot of the pressure data is shown in FIG. 1B. The pressure sensor matrix may be calibrated to exclude data from pressure sensors that measure less than a threshold pressure. The threshold may correspond to, for example, ambient air pressure. The pressure sensors that measure a pressure above the threshold may be considered active sensors, located in an area of the pressure sensor matrix on which a test participant is standing.

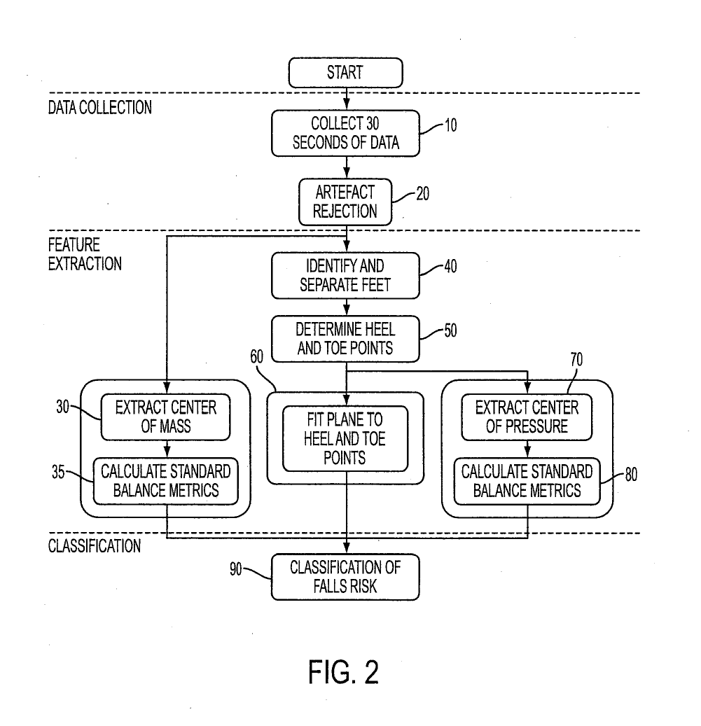

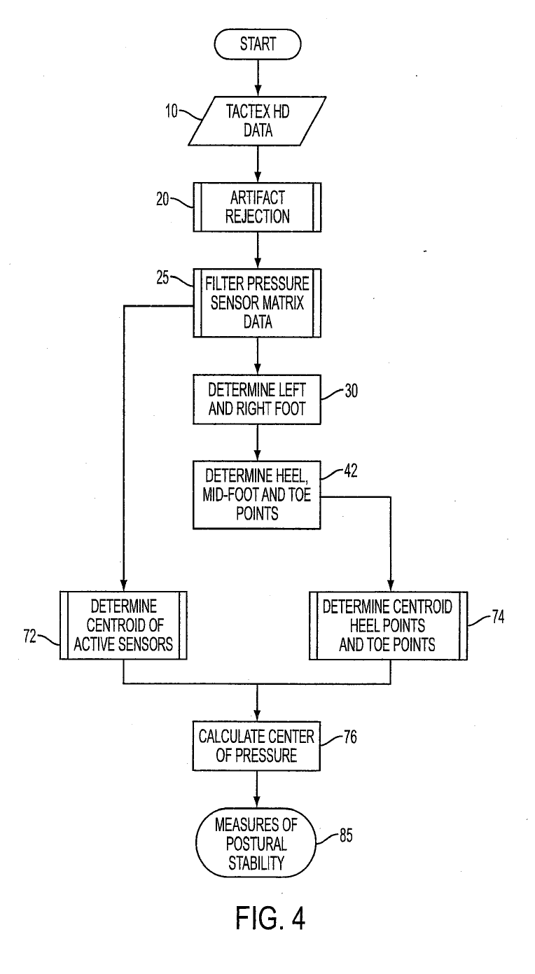

To gauge a participant's postural stability and risk of falling, the pressure data generated from the pressure sensor matrix may be used to calculate, for example, changes in the person's center of mass and center of pressure while standing. FIG. 2 illustrates an example overview of operations in such a falls risk assessment technique. At operation 10, pressure sensor data is collected for an interval of 30 seconds. The interval may be shorter, such as for a few seconds, or longer, such as for a few minutes. The data collection may take place at the beginning, middle, end, or some other interval of a balance test. At operation 20, artefact rejection may be performed to remove spurious data. For example, a spike in measured pressure values may be rejected, or a series of pressure values exhibiting large fluctuations may be rejected. The pressure data values may also be associated with video data to identify times during which, for example, a test participant was not standing still on the pressure sensor matrix. Pressure data during those times may be excluded. The data may also be filtered, such as shown in the flow diagram in FIG. 4. The filtering may involve determining if a sufficient number of pressure sensors are active to adequately relate pressure data to postural stability and may be high pass filtered to remove noise.

One of the metrics that may be derived in the balance assessment algorithm is a test participant's center of mass. At operation 30, the center of mass (COM) may be calculated based on the pressure exerted along the pressure sensor matrix. In one example, the COM may be calculated as

$$COM = \frac{Σ {m}_{i} {r}_{i}}{Σ {m}_{i}} ,$$

where mᵢ is the pressure applied at each coordinate rᵢ of the pressure sensor matrix.

At operation 35, the COM data may be used to calculate, either by itself or along with center of pressure (COP) data and/or heel and toe points data, standard balance metrics. For example, the COM data may be used to calculate sway length, COP velocity, area, and frequency measures.

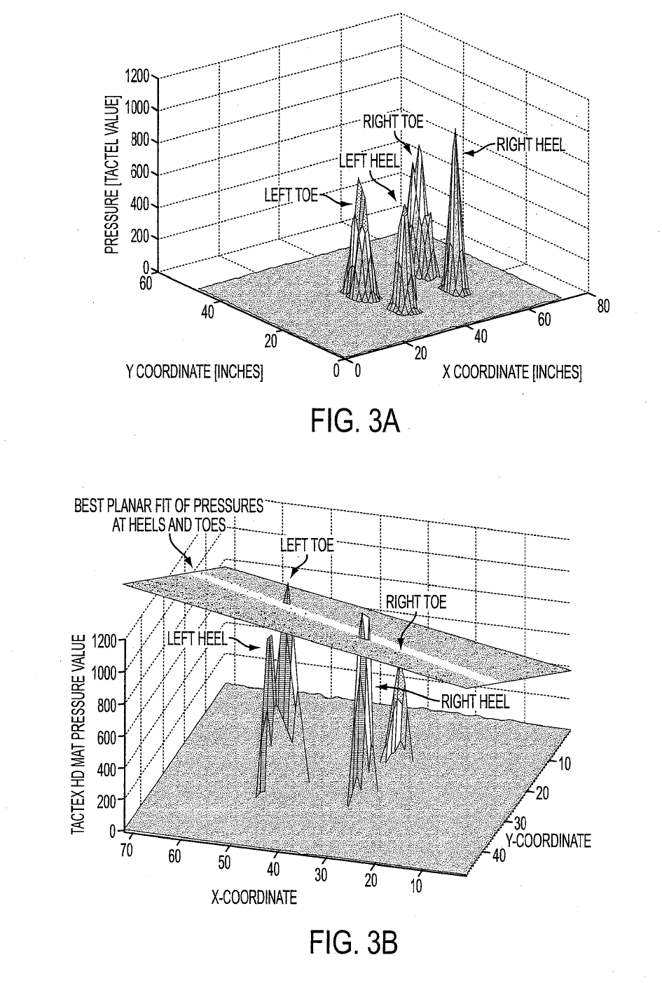

The balance assessment algorithm may also differentiate between pressure points exerted by a participant's left foot and pressure points exerted by the participant's right foot. For example, at operations 40 and 50, the pressure points generated by a test participant may be associated with the left and right feet of the participant as well as with the heels and toe points of the participant. For example, to localize the heel and toe points of each foot, each frame of pressure sensor data may first be scanned horizontally from left to right across each feet. The first active pressure coordinates registering pressure may be defined as the outer edge of the foot. The foot may be empirically defined as having a maximum width that spans, for example, eight pressure coordinates (e.g., 10.1 cm). The inner and outer edges of both feet may be located based on the empirically defined maximum feet width. For each feet, a toe point and a heel point may be located. For example, once an approximate area of the heel and toe for a foot is located, the highest local pressure point may be located through an iterative search. The coordinate of the highest local point in the toe area may be defined as the toe point, and the coordinate of the highest local point in the heel area may be defined as the heel point. FIG. 3A shows example pressure values that may be used to identify the toe and heel points from a test participant.

The balance assessment algorithm may also derive parameters correlating to a planar fit of toe, heel, and/or mid-foot points of a test participant's feet. For example, at operation 60, a planar fit may be performed on the four points corresponding to the toe and heel points of the two feet of the test participant. FIG. 3B shows a closest fit 2-dimensional plane of the four points (left toe point, left heel point, right toe point, and right heel point). In one example, the closest fit plane may be defined as

$$z = \frac{A}{C} x + \frac{B}{C} y + \frac{D}{C} ,$$

where D/C represents the overall pressure placed upon the pressure sensor matrix, A/C represents the left-right difference in pressure placed upon the pressure sensor matrix, and B/C represents the up-down difference in pressure placed upon the pressure sensor matrix.

The balance assessment algorithm may also derive the center of pressure and parameters related to the center of pressure for a test participant. For example, at operation 70 and 80, the center of pressure may be calculated and used to calculate standard balance parameters such as sway length, COP velocity, area, and frequency measures. The parameters may also include the mean distance between each COP point and a mean COP point, the root mean squared distance between each COP point and the mean COP point, the total COP path length travelled over the recording period, and the average velocity of the COP. The measures may include any other measures related to balance or postural stability. At operation 72, the center of pressure may be calculated based on the average position of all active sensors, which may include all sensors in the matrix that experienced a pressure above a baseline threshold. The baseline threshold may be set at zero, for example, or at a level that represents pressure experienced by the pressure sensor matrix when a test participant is not standing on the matrix. For example, the COP may be an average coordinate of all pressure sensor coordinates at which the measured pressure exceeds ambient air pressure, and may be a weighted average that moves the COP closer to coordinates measuring higher pressure values.

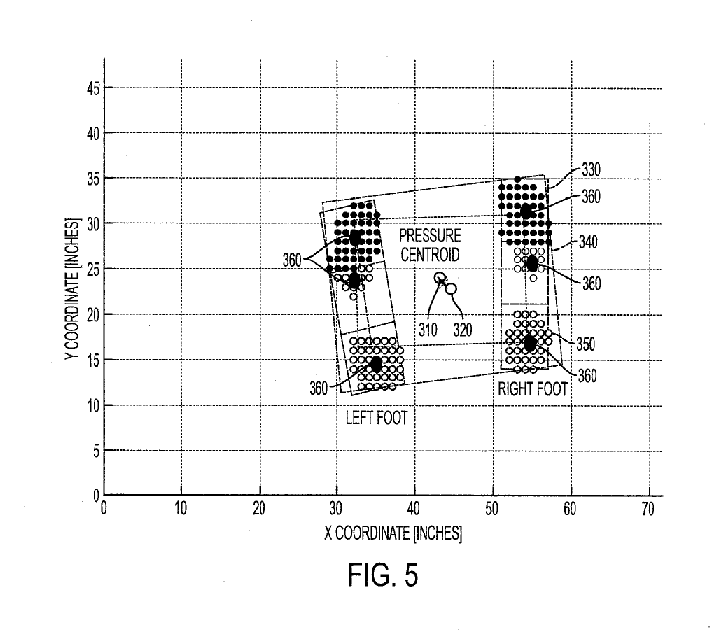

At operation 74, the COP may be calculated based on an average of regional pressure centroids. At operation 74, a centroid of the pressure points may be calculated for each of a heel region and toe region of the two feet that were identified at operation 30. The COP of the test participant may be calculated as the average of the four regional centroids. In this embodiment, a mid-foot region and a centroid of the pressure points of the mid-foot region may also be identified from among the pressure sensor points. The COP may also be based on an average that includes the centroid of the pressure points of the mid-foot region. Calculation of the COP is further illustrated in FIG. 5.

FIG. 5 illustrates a graphical depiction of one snapshot of pressure data. The figure shows a pressure sensor matrix able to detect pressure caused by the toes and heels of a user standing on the sensor matrix. Each point in FIG. 5 represents a coordinate where a threshold pressure was detected, and may also indicate a measured value of the detected pressure. For example, the sensors may be able to detect and quantify the greater amount of pressure exerted by the right heel compared to the left heel. The data in FIG. 5 may be collected by as many as tens or hundreds of thousand of sensors or as few as four pressure sensors.

Each snapshot of pressure data may be processed to calculate the COP for the snapshot. The COP may refer to the geometric center, average, or any coordinate representative of the pressure data of the snapshot. In one embodiment, the COP may be an average position of all the coordinates on the sensor matrix where pressure was detected. The average may be weighted to move the COP closer to coordinates that measured higher pressure.

The COP may be based on regional pressure centroids associated with a toe and heel of each feet. For example, FIG. 5 shows the result of analysis that divides pressure sensor coordinates of a snapshot into regions corresponding to pressure from a left foot and regions corresponding to pressure from a right foot. For the right foot, for example, the coordinates may be divided into a toe region 330, a mid-foot region 340, and a heel region 350. A centroid of the pressure points 360 may be calculated for each of these regions. For example, a regional centroid of the pressure points for toe region 350 may be calculated by averaging all the coordinates in region 350. The COP for the snapshot may be calculated as the average of some or all of the regional pressure centroids. For example an average of the coordinates of the four toe and heel region pressure centroids may be calculated. The average may be weighted, with different regional pressure centroids having different pressure values. FIG. 5 shows a COP 310 calculated as an average of all coordinates, in accordance with operation 72. Also shown is the COP 320 calculated as an average of the positions of regional centroids, in accordance with operation 74. The COP's calculated from the two techniques may yield different coordinates, or may yield the same coordinates.

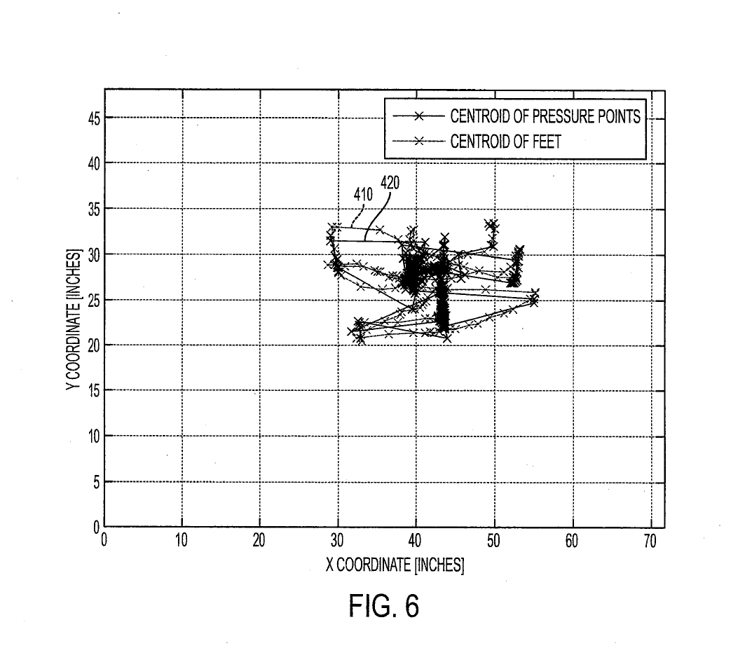

At operation 85, the plurality of COP's from a plurality of snapshots collected by the sensor matrix may be used to analyze the movement, balance, and/or posture of the test participant. FIG. 6 shows a graphical view of the plurality of COP's across a plurality of snapshots. Plot 410 shows the COP's calculated from averaging all the active pressure sensor coordinates, in accordance with operation 72. Plot 420 shows the COP's calculated from averaging regional pressure centroids, in accordance with operation 74. The COP's from the snapshots may be used to derive a general pattern of movement of a user, changes in balance that causes the shifts in pressure from snapshot to snapshot, or any other metric related to balance and postural analysis.

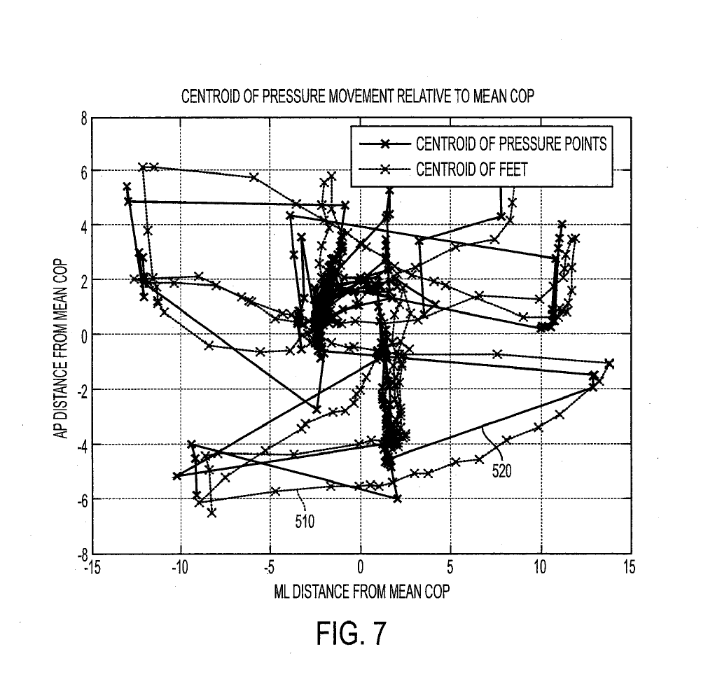

Various metrics relating to the COP and changes in the COP may be calculated to analyze a participant's postural stability. FIG. 7 illustrates example COP values measured along an anteroposterior (AP) direction and along a mediolateral (ML) direction. The plurality of points on the figure represents the plurality of distances calculated from a plurality of snapshots. Plot 510 shows the AP and ML components of the mean of the COP's calculated from averaging all of the active coordinates of the pressure sensor matrix. Plot 520 shows the AP and ML components of the mean of the COP's calculated from averaging the regional pressure centroids of the toe and heel regions. The figure shows that the COP's may be located within a range around an average COP. The plot of the COP's may reflect shifts in a test participant's COP due to, for example, shifts or loss in balance. A greater amount of deviation of the mean COP from the AP and ML axes may indicate less postural stability.

The center of pressure data may be used to also calculate a mean distance, MDIST, between each COP point and the mean COP point:

$$MDIST = \frac{1}{N} Σ RD [ n ] = \frac{1}{N} Σ \sqrt{( {AP [ n ]}^{2} + {ML [ n ]}^{2} )} ,$$

where AP and ML are COP coordinates relative to a mean COP, and are used to calculate the Euclidean distance for each snapshot, RD[n], from each set of the coordinates relative to the mean COP point. By relating the location of the COP to the mean COP, a more standardized center of pressure (COP) time series may be obtained, which can be used in tandem with standard time and frequency domain measures of postural stability to evaluate balance under a variety of conditions.

The COP coordinates relative to the mean COP may be calculated as:

AP[n]=APo[n]− A̅P̅, where APo[n] represents the anteroposterior time series coordinates of the COP and where A̅P̅ is the mean anteroposterior (AP) COP coordinate over the period in which the pressure data is recorded.

ML[n]=MLo[n]− M̅L̅, where MLo[n] represents the mediolateral time series coordinates of the COP and where M̅L̅ is the mean mediolateral (ML) COP coordinate over the period in which the pressure data is recorded.

The mean AP and ML coordinates may be calculated as:

$$\overline{AP} = \frac{1}{N} Σ APo [ n ]$$

$$\overline{ML} = \frac{1}{N} Σ MLo [ n ]$$

The COP data may also be used to calculate a root mean squared distance between each COP point and the mean COP point:

$$\overline{RDIST} = \sqrt{\frac{1}{N} Σ {RD [ n ]}^{2}}$$

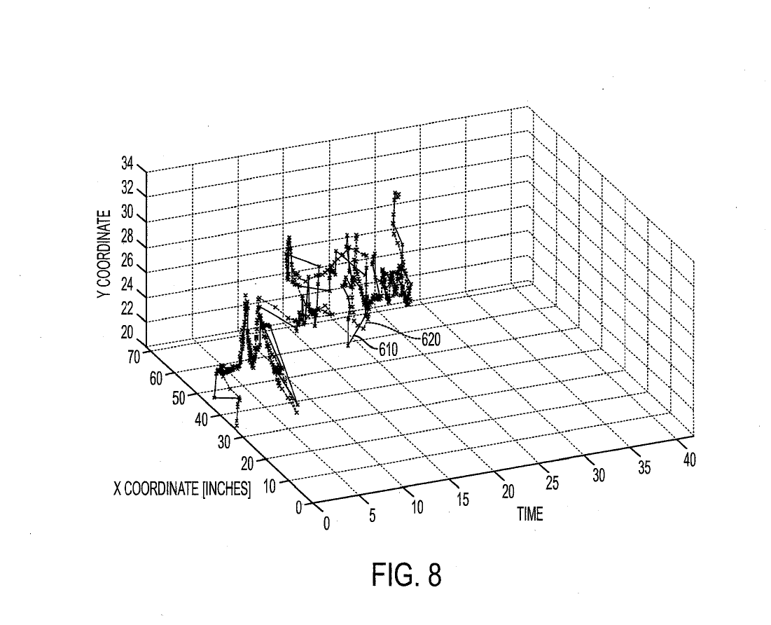

The balance assessment algorithm may also analyze how the COP varies over time. FIG. 8 depicts COP's from a plurality of snapshots. Plot 610 shows the time-based shift in the COP's calculated from averaging all active points of the pressure sensor matrix, in accordance with operation 72. Plot 620 shows the time-based shift in the COP calculated from averaging the regional pressure centroids of the heel and toe regions, in accordance with operation 74.

A total COP path length, TOTEX, travelled over the recording period may be calculated as

$$TOTEX = {\sum }_{n = 1}^{N - 1} \sqrt{Diff\_AP {( n )}^{2} + Diff\_ML {( n )}^{2}} ,$$

where Diff_AP(n) and Diff_mL(n) represents the change in the COP coordinates during the recording period:

Diff_{—}AP(n)=AP(n+1)−AP(n)

Diff_{—}ML(n)=ML(n+1)−ML(n)

The average velocity of the COP, MVELO, may be calculated as

MVELO=TOTEX/T

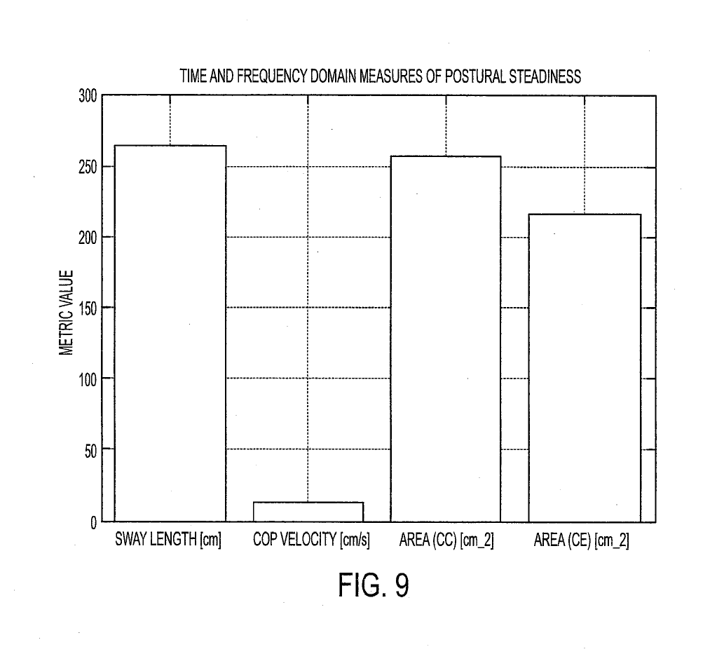

Other metrics, including time and frequency domain measures relating to balance and postural stability, may be calculated at operation 190. For example, FIG. 9 shows example sway length, center of pressure (COP) velocity, area (CC), and area (CE) of a 28-year old test participant weighing about 150 lbs and 6 feet.

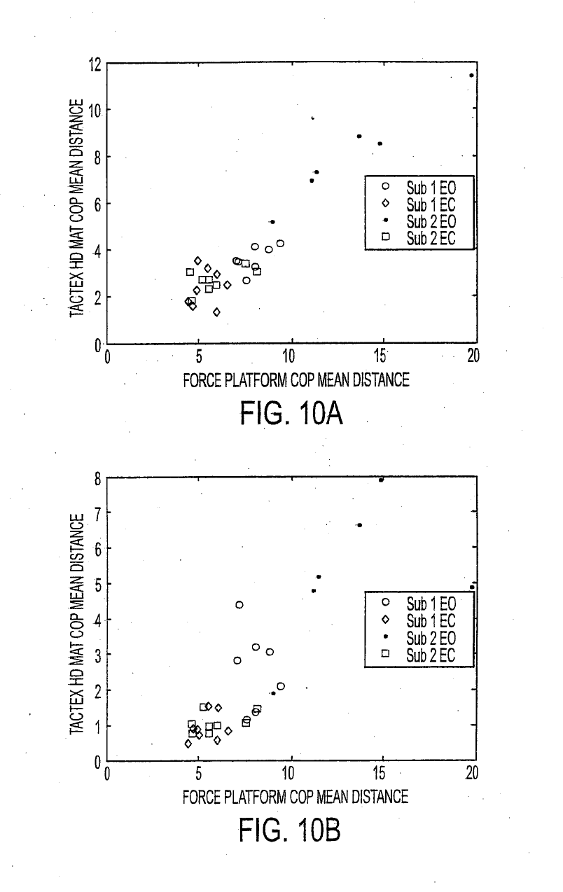

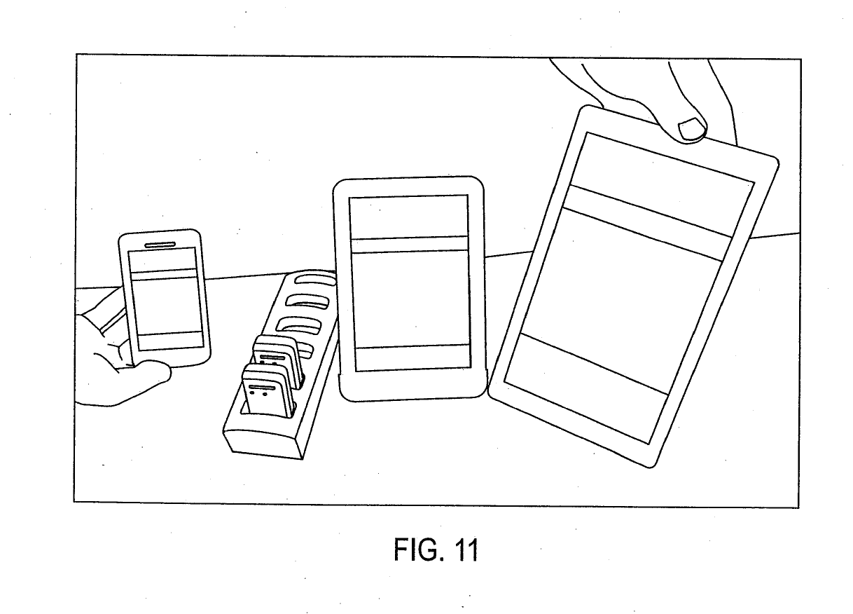

For verification of pressure data from a pressure sensor matrix, the pressure data collected by the matrix may be compared against those collected by a force plate. The COP mean distance, for example, derived from the force plate versus that derived from the sensor matrix may be compared. For example, FIG. 10A shows example COP mean distance values measured using a force plate and measured using a pressure mat. The COP data in FIG. 10A are calculated as an average of all active sensor points. The data may be simultaneously generated for both the force plate and pressure mat by placing the pressure mat flush on top of the force plate. FIG. 10A shows COP mean distance calculations from four sets of balance tests, divided among two test subjects (e.g., one 29-year, 80 kg male and one 22-year, 50 kg male). Each test subject performed a set of balance tests with eyes open and a set of balance tests with eyes closed. FIG. 10B shows COP mean distance in which the COP data is calculated from averaging regional pressure centroids of the heel and toe regions. The force plate calculations and pressure mat calculations may be used to validate the pressure data obtained from the pressure mat. If there is too much variation in the pressure mat data, the pressure data may be excluded from analysis, or may be reacquired. For example, if pressure data collected by the pressure mat during an “eyes-open” test shows too much variation or does not correlate well with pressure data collected by the force plate, the test participant may be asked to repeat the test with eyes closed.

The COP's may be calculated using any hardware platform operable to receive data from a pressure sensor matrix. In one example, a BioMOBIUS™ platform may be used. The platform may provide a graphical user interface for a user or a clinician, real time processing of the pressure data, and support for predefined sensor types, such as the SHIMMER® sensor. The results could be analyzed by a clinician, or could contain a real-time video link simultaneously with data capture which would allow clinical personnel to observe and direct patients in their homes while performing directed balance routines.

The balance and postural analysis may combine pressure and kinematics with other data. The data may be analyzed as part of a quantitative balance assessment tool that could be used for falls risk assessment. For example, the pressure sensor matrix may calculate a user's weight from pressure data. An analysis processor or processors may adjust a user's postural or balance assessment based on user weight. The analyzed data may also be combined with other clinical information such as age, gender, height etc to assess the risk of falls for the user. The tool may be used in a clinical setting, or may be used in a home setting. A real-time video link may be incorporated with the standing balance assessment to feed observations of a test participant to a remote location. The video link allows clinicians to observe patients performing tests in their homes. The clinicians may use the video data to neglect invalid trials.

A falls risk model may correlate the postural and balance metrics described herein, such as gait length, sway length, centroid velocity, center of mass, and/or other pressure-related parameters with a risk of fall. The models may also incorporate variables derived from kinematic sensor measurements. Pattern recognition, for example, may be used to generate classifier models for falls risk assessment. The pattern recognition analysis may employ a supervised pattern recognition approach that uses a training set to generate classifier models. For example, in a retrospective approach to assessing falls risk, a training set may comprise self-reported falls history of the test participants. The classifier model may be trained to better match the self-reported falls history of the test participants who generated the postural and balance metrics. In a prospective (predictive) approach to assessing falls risk, a training set may comprise sensor data obtained at the original assessment and data on falls that test participants experienced in the period after the balance assessment test. The data may be collected by following up with test participants in a period (e.g., two years) after their balance assessment test. This prospective approach may yield more accurate classifier models because the self-reported history used in a retrospective approach may be less reliable than falls data gathered from test participants after their test. The supervised pattern recognition may be performed with techniques such as discriminant analysis, neural networks, support vector machines, naïve bayes classifiers, or any other supervised pattern recognition algorithm.

The pattern recognition analysis may group the metrics into vectors of features and identify features that should be included in a falls risk model. For example, filter techniques of a feature selection method may rely on general characteristics of the metrics, such as correlation with class labels, to evaluate and select the feature subsets. Wrapper techniques of the feature selection method may assess the performance of a classifier model on given datasets to evaluate each candidate feature subset. Wrapper methods may search for a more optimal feature set for a given model. A wrapper method, such as sequential forward feature selection, may sequentially add features to an empty set until the addition of further features does not increase the classification accuracy.

Other pattern recognition techniques, such as unsupervised learning, may also be used. Unsupervised learning may attempt to find inherent patterns in the pressure-derived parameters of the balance assessment test. The unsupervised pattern recognition may be performed with techniques such as K-means clustering, hierarchical clustering, kernel principal component analysis, or any other unsupervised pattern recognition algorithm.

Use of classifier models is discussed more in U.S. patent application Ser. No. 13/186,709, entitled “A Method for Body-Worn Sensor Based Prospective Evaluation of Falls Risk in Community-Dwelling Elderly Adults,” which is herein incorporated by reference in its entirety.

Other falls prediction methods, such as logistic regression, may also be used. Generating logistic regression models is discussed more in U.S. application Ser. No. 12/782,110, entitled “Wireless Sensor Based Quantitative Falls Risk Assessment,” the entire content of which is incorporated herein by reference.

Those skilled in the art will appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

What is claimed is:

1. A method of assessing falls risk, comprising:
receiving a first plurality of detected pressure points, each detected pressure point associated with one of a set of coordinates of a pressure sensor matrix;
associating a first set of coordinates of the pressure sensor matrix with a first foot region based on the first plurality of detected pressure points;
calculating a first pressure centroid for the first foot region;
associating a second set of coordinates of the pressure sensor matrix with a second foot region based on the first plurality of detected pressure points;
calculating a second pressure centroid for the second foot region;
calculating a center of pressure based on the first pressure centroid and second pressure centroid; and
generating a balance assessment metric based on the calculated center of pressure.

2. The method of claim 1, further comprising receiving a second plurality of detected pressure points at a time different from a time of receipt of the first plurality of detected pressure points, each detected pressure point associated with one of the set of coordinates of the pressure sensor matrix; and
calculating a second center of pressure based on the second plurality of detected pressure points.

3. The method of claim 2, further comprising generating a falls risk assessment model based on the first center of pressure and the second center of pressure.

4. The method of claim 2, wherein the balance assessment metric comprises a sway length, gait length, a velocity of the center of pressure, a mean distance between each center of pressure point over a plurality of snapshots and the mean center of pressure point during the plurality of snapshots, a root-mean-squared distance between each center of pressure point during the plurality of snapshots and the mean center of pressure point during the snapshots, a total center of pressure path length travelled over a plurality of snapshots, or any combination thereof.

5. An apparatus for assessing risk of falls, comprising:
a matrix of pressure sensors configured to generate data identifying a first plurality of pressure points exerted on the matrix of pressure sensors, each of the pressure sensors associated with one of a plurality of coordinates of the matrix; and
a processor configured to:
receive the first plurality of pressure points from one or more sensors of the matrix of pressure sensors,
associate a first set of coordinates of the matrix with a first foot region based on the plurality of pressure points,
calculate a first pressure centroid for the first foot region,
associate a second set of coordinates of the matrix with a second foot region based on the first plurality of pressure points,
calculate a second pressure centroid for the second foot region,
calculate a center of pressure based on the first pressure centroid and second pressure centroid, and
generate a balance assessment metric based on the calculated center of pressure.

6. The apparatus of claim 5, wherein the processor is further configured to receive a second plurality of pressure points at a subsequent time, and to calculate a second center of pressure based on the second plurality of pressure points.

7. The apparatus of claim 6, wherein the processor is further configured to generate a falls risk assessment model based on the first center of pressure and the second center of pressure.

8. The apparatus of claim 6, wherein the balance assessment metric comprises a sway length, a gait length, a velocity of the center of pressure, a mean distance between each center of pressure point over a plurality of snapshots and the mean center of pressure point during the plurality of snapshots, a root-mean-squared distance between each center of pressure point during the plurality of snapshots and the mean center of pressure point during the snapshots, a total center of pressure path length travelled over a plurality of snapshots, or any combination thereof.

9. The apparatus of claim 5, wherein the apparatus comprises a mat embedding the plurality of pressure sensors.

10. A system for assessing risk of falls, comprising:
a matrix of pressure sensors configured to generate data identifying a first plurality of pressure points exerted on the matrix of pressure sensors, each of the pressure sensors associated with one of a plurality of coordinates of the matrix; and
a processor configured to:
receive the first plurality of pressure points from the matrix of pressure sensors,
associate a first set of the plurality of coordinates of the matrix with a first foot region based on the first plurality of detected pressure points,
calculate a first pressure centroid for the first foot region,
associate a second set of the plurality of coordinates of the matrix with a second foot region,
calculate a second pressure centroid for the second foot region,
calculate a center of pressure based on the first centroid of the pressure points and the second pressure centroid, and
generate a balance assessment metric based on the calculated center of pressure.

11. The system of claim 10, wherein the processor is further configured to receive a second plurality of pressure points at a subsequent time from the matrix of pressure sensors, and to calculate a second center of pressure based on the second plurality of detected pressure points.

12. The system of claim 11, wherein the processor is further configured to generate a falls risk assessment model based on the first center of pressure and the second center of pressure.

13. The system of claim 11, wherein the balance assessment metric comprises a sway length, a gait length, a velocity of the center of pressure, a mean distance between each center of pressure point over a plurality of snapshots and the mean center of pressure point during the plurality of snapshots, a root-mean-squared distance between each center of pressure point during the plurality of snapshots and the mean center of pressure point during the snapshots, a total center of pressure path length travelled over a plurality of snapshots, or any combination thereof.

14. The system of claim 10, wherein the matrix of pressure sensors comprises a pressure sensor mat embedding the matrix of pressure sensors.

15. A method for assessing falls risk, comprising:
receiving a first plurality of detected pressure points at a first instance in time, each of the first plurality of detected pressure points associated with one of a first set of coordinates of a pressure sensor matrix;
calculating a first center of pressure associated with the first plurality of detected pressure points;
receiving a second plurality of detected pressure points at a second instance in time, each of the second detected pressure points associated with one of a second set of coordinates of the pressure sensor matrix;
calculating a second center of pressure associated with the second plurality of detected pressure points;
calculating a mean distance between a mean center of pressure and at least the first and second centers of pressure, a root-mean-squared distance between the mean center of pressure and at least the first and second centers of pressure, a total center of pressure path length for at least the first and second centers of pressure, an average velocity of the center of pressure for at least the first and second centers of pressure, or any combination thereof.
